(19) United States
(12) Patent Application Publication
(10) Pub. No.: US 2003/0219429 A1
(43) Pub. Date: Nov. 27, 2003

(54) Composition and method for bone regeneration

(75) Inventor: John Arnold Budny, Westlake Village, CA (US)

Correspondence Address: Colin P. Abrahams, Suite 400, 5850 Canoga Avenue, Woodland Hills, CA, 91367, US

(73) Assignee: Pharmacal Biotechnologies, LLC

(21) Appl. No.: 09/991,588

(22) Filed: Nov. 21, 2001

Related U.S. Application Data
(63) Continuation-in-part of application No. 09/122,348, filed on Jul. 24, 1998, now abandoned.

(51) Int. Cl. A61K 38/48; A61K 9/14

(52) U.S. Cl. 424/94.63; 424/486

(57) ABSTRACT

A composition for modulating bone regeneration composition comprises a matrix selected from the group consisting of glycolic acid, lactic acid, collagen, demineralized bone, or a combination thereof. A first biologically active molecule comprising a fibronectin is attached to a portion of the matrix, to facilitate osteoblast activity and for promoting an increase in bone formation. A second biologically active molecule comprising a vitronectin, selected for its ability to attract osteoclasts and produce an inhibiting effect on osteoclast activity to thereby promote a decrease in bone resorption, is also attached to a portion of the matrix.

### CROSS REFERENCE TO RELATED APPLICATIONS

This application is continuation in part of U.S. patent application Ser. No. 09/122,348 filed Jul. 24, 1998, which is incorporated herein by reference in its entirety.

### FIELD AND BACKGROUND OF THE INVENTION

This invention relates to compositions and methods which facilitate bone regeneration and healing.

There is an ongoing need to replace, modify or correct defects which may be caused or arise in osseous tissue. Usually, when osseous tissue requires replacement, modification or some other form of functional correction, the cause thereof may fall into one of two categories. The first such category relates to those circumstances resulting from disease conditions or states, and the second is a consequence of some traumatic event. While the need for hard tissue repair is diverse, the various ameliorating procedures available for such repair are similar in approach. Replacing, reconfiguring, repairing and reconstructing bone tissue involves complex and often difficult procedures that may have permanent consequences, and may even require the patient to alter his or her activities after an orthopaedic correction event.

Bone disease is one of the major causes and conditions which give rise to the necessity for osseous tissue modification. An example of such a disease is scoliosis. The osseous tissue modification may also be necessary due to congenital malformations, namely, inborn errors of metabolism, which cause abnormal bone development or abnormal skeletal use. Replacement or reconstructive surgery may be necessary to correct these defects. Other bone diseases requiring correction or reconstruction of bone tissue include serious localized bone resorption, a prime example of which is periodontal disease. This condition may require bone replacement to achieve normal functional activity. Planned osseous tissue removal, such as bone tumor excision, will also require replacement and reconstruction of the area from which the cancerous tissue was removed.

A traumatic event may result in the need to repair or reconstruct bone. The trauma may be chronic, such as bone wearing or spinal fusion with age (osteoarthritis), or it may be acute, such as with bone fractures. The severity of the traumas will, of course, determine whether reconstructive surgery or some other form of bone reconstruction is required. In fractures which are less serious, and do not require surgery, there is a distinct economic advantage to decreasing the healing time, and reducing the time that immobilization of a fracture requires for adequate fusion of the fracture.

There are a significant variety of standard tools and procedures available to the orthopedist for effecting bone reconstruction. Over the years, these various tools have undergone improvements and refinements. The standard methods for bone reconstruction can basically be classified into three categories: mechanical, physiological and pharmacological.

Probably the most well-established method for bone repair is the mechanical one, and this typically involves hard implants and hardware, such as plates, pins and screws. Within the category of hard implants, there exist an array of plastics, organic-based synthetic cements and metal prostheses. There are two major considerations and concerns in using mechanical hardware and implants. The first relates to the effectiveness of the physiological integration of the hardware into the body systems, while the second is that of the long-term durability of the non-biological material which has been implanted. Despite these problems, mechanical implants are very popular, and, while not comprising living bone tissue, make significant contributions assisting in the bone reconstruction.

More recently, a physiological approach has been used to facilitate bone reconstruction. This approach uses bone grafting, i.e., the transplant of bone material to the diseased or trauma site from a non-affected source. There are two types of grafting, namely, autograft and allograft.

Autografting, which involves the use of bone for transplant from the person requiring the bone tissue, essentially uses two surgical procedures. The first procedure harvests the transplantable bone sample; the second procedure is the actual implantation of the bone sample at the repair site. Depending on the circumstances and the extent of the repair that is necessary, there may well be a limitation of the amount of transplantable bone tissues available, since the quantity of such bone tissue required for the repair process may exceed the supply. This is because autografts involve transplanting bone tissue that is harvested from a remote site within the host to the area of osseous tissue on the host that is being repaired.

In contrast, allograft is the transplantation of bone tissue from a donor, or cadaver, to the repair site on a recipient. This is a procedure that appears to be gaining popularity, and bone as a transplantable biologic sample is second only to blood transfusion. Allograft procedures do, however, have drawbacks. The two most notable issues associated with allograft repair of osseous tissue are infectious disease transmission and immunological incompatibility. On the other hand, allografting has the distinct advantage of eliminating one of the two surgical procedures required in the autografting technique, and the supply of transplantable bone tissue is usually larger than if the sample were derived solely from the host.

The third approach in osseous tissue therapy is pharmacological. Pharmacological treatment involves the administration of therapeutic agents to treat primarily bone diseases. One of the drawbacks of using pharmacological agents is that, because of the systemic nature of these therapeutic agents, their use in site-specific bone replacement, reconfiguration or reconstruction is limited. However, in certain circumstances, the pharmacological approach using such agents as diphosphates is useful for treating system-wide bone diseases affecting the body at multiple sites, a common example of which is osteoporosis.

The discussion above relates to mechanical, physiological and pharmacological approaches which are fairly standard tools used in bone reconstruction and repair. However, over the years, there has also developed a wide variety of non-standard, research-oriented approaches for addressing site-specific osseous tissue replacement, reconfiguring and reconstruction. While many of these approaches have little or no current clinical application or practice, they do provide promising avenues for future therapies. The common thread in these new approaches is two-fold: the first requires the development of new osseous tissue that is indistinguishable from the host's normal bones, while the second uses some sort of osseous or osseous-like implantation. The nature of these non-standard approaches can conveniently be grouped into four basic classes according to the type of compositions and/or materials used. These are: (i) natural compositions and components, for example, using demineralized bone, and collagen/mineral mixes; (ii) semi-synthetic compositions and components, which use, for example, modified coral to produce a hydroxyapatite-like implant (hydroxyapatite, as will be discussed below, is the main mineral component of bone tissue); (iii) site-specific therapeutic treatment such as, for example, localized administration of bone morphogenetic proteins (BMPs); and (iv) scaffolding materials, either with or without additives. Importantly, researchers have been able to induce ectopic bone formation using demineralized bone tissue, an observation which resulted in a greater understanding of the role played by the organic matrix, or the extra-cellular matrix (ECM) in bone physiology as well as non-osseous tissue repair. Developments to induce bone tissue healing have been researched using combinations of mineral mixes along with collagen, milled bone combined with extracted bone proteins, and the use of ceramic fibers.

Possibly the most significant effect which has resulted from this non-standard approach to bone reconstruction is the realization of the importance that the extracellular matrix has on tissue repair and regeneration. Research in the area of tissue engineering has expanded significantly with advances in synthetic scaffolding materials, which are typically used as implants. Some of these synthetic scaffolds may be specifically designed to be durable, while others may be specifically designed to erode or dissolve over time.

Furthermore, the substantial role of various proteins in tissue repair has been clearly identified, and the realization of the importance of such proteins has followed closely upon the recognition and acceptance of the importance of the organic matrix and its components in bone tissue repair. Such components include crude extracts, gross factors, BMPs, fibronectin and other extracellular matrix proteins. While these “other” proteins may contribute in some measure to providing a structure or framework for cellular proliferation in the tissue repair and regeneration process, their primary functions lie elsewhere. On the other hand, extracellular matrix proteins, glycoproteins, and the like, which continue to be identified as research proceeds, perform crucial functions related to cell adhesion, regulation, and both inter- and intra-cellular communication.

### SUMMARY OF THE INVENTION

In one aspect, the invention is a composition and method for facilitating osseous tissue healing or increased bone accumulation resulting from fractures or bone losses. The composition and method of the invention simultaneously increases bone formation and at the same time decreases bone resorption. This simultaneous regulation of opposing objectives and net accumulation of bone is accomplished by encouraging osteoblastic activity and at the same time discouraging osteoclastic activity.

In yet a further aspect, the composition contains a biodegradable or resorbable component as a means to retain the appropriate biologically active molecules that encourage bone formation (osteoblastic activity) and discourage bone resorption (osteoclastic activity).

Bone tissue is unique in vertebrates, providing both structure and support for the organism, while being a reservoir for minerals such as calcium, magnesium, phosphorus and sodium. Unlike other tissues, such as soft tissues, found in living bodies, a considerable portion of bone material—approximately 70%—is an inert, inorganic, mineral-based substance. The remaining approximately 30% of the total bone tissue comprises an organic component. Together, these diverse classes of materials provide a structure and function that complement each other and are, at the same time, integrated into other physiological systems of the total organism. Due to the vascular structures contained within bone material, the bone receives about 10% of the cardiac blood flow output.

As mentioned, bone is made up of mineral (inorganic) and organic components. The mineral portion of bone consists principally of hydroxyapatite, which is a crystalline form of calcium and phosphate ions. The mineral portion of bone also includes other less crystalline composites, which tend to give bone its amorphous appearance, and these include magnesium, sodium, potassium and other less prevalent cations. The most abundant non-phosphate anion is carbonate.

The organic portion of the bone tissue can be broken down into its collagen fiber component, “ground substance”, and non-collagenous proteins. The collagen fibers constitute the predominant portion of the organic component, and make up 90-95% thereof. The remaining 5-10% is made up of the ground substance and new collagenous fibers. The ground substance has as its major components the extra cellular fluid and proteoglycans. The most predominant proteoglycans are chondroitin sulfate and hyaluronic acid. In addition, the organic portion or matrix contains a wide variety of other important non-collagenous proteins such as fibronectin, osteopontin, osteocalcin, osteonectin, thrombospondin and other, less characterized proteins. While these proteins individually comprise less of the organic matrix than collagen or proteoglycans, they perform vital functions in the area of cell adhesion, intra- and inter-cellular communication and other necessary physiologic functions.

The complementary composition of mineral and organic matter gives bone tissue a dual mechanical durability. On the one hand, bone tissue has a significant compression strength, derived from the mineral component, and, on the other hand, possesses tensile strength derived from its organic component. These properties constitute the “mechanical” portion of the “biomechanical” characteristics and features of bone tissue. Further, it should be appreciated that, even though the major portion of bone is comprised of inorganic components, it is not by any means an inert substance. The “bio” or living aspects of bone's “biomechanical” property traits are a critical feature of bone, and contribute to the dynamic qualities thereof. Bone is a living tissue, containing living cells. Under the direction and influence of these cells, the inorganic or mineral portion of the bone is constantly being removed by a process known as resorption, as well as replaced by a process known as deposition. This dynamic process of continually removing and replacing bone by resorption and deposition is generally called bone remodelling, and is under the direct and balanced influence of two bone cell types: osteoblasts and osteoclasts.

Osteoblasts are the types of cells that are responsible for the formation of new bone. These cells, during their productive bone forming cycle, are found at the exterior portion of the mineral surface in layers just below the fibrous periosteum, which covers the bone surface. As new bone is laid down, namely, when neocalcification occurs, the osteoblasts are encapsulated by collagen fibers. These collagen fibers are in fact secreted by the osteoblasts. The osteoblasts, when encapsulated by the collagen fibers, become part of the osteoid, which is the uncalcified bone matrix. When an osteoblast is completely engulfed within an osteoid, the osteoblasts become osteocytes, a bone cell that is characteristic of adult bone and which is isolated in a lacuna of the bone substance. The collagen fibers that are secreted serve as the matrix and structure for calcium and phosphate to crystallize into hydroxyapatite, the predominant mineral portion of bone tissue.

Osteoclasts, on the other hand, are responsible for bone resorption. As might be expected, osteoclasts are typically found within the mineral portion of the bone. Osteoclasts are multi-nuclear, phagocytes which are originally derived from monocytes in bone marrow. The process of bone resorption under the influence of the osteoclasts appears to involve a two-step process. The first step is the dissolution of the minerals by secreted organic acids, and the second is the dismantling of the organic matrix by the hydrolytic enzymes which are also secreted by the osteoclasts. The process of bone remodelling, or turn-over, is the process of bone removal from the interior of the bone, while at the same time laying down new minerals which occurs at the surface of the bone. Each of these processes is carried out by specific cell types, namely, bone removal by osteoclasts, and the laying down of new bone by osteoblasts.

The simultaneous processes of resorption and deposition are regulated by diverse control mechanisms. These include a broad array of regulatory mechanisms such as hormones, vitamins, biologic response modifiers and cytokenes which are immuno-regulatory substances secreted by cells of the immune system. These various agents involved in the regulatory process act both systemically and locally to maintain the health and desired balance between the osteoblastic and osteoclastic activities. When the regulatory and control factors are distorted or fail, various disease conditions result.

While it is important that the regulatory and control factors ensure the desired balance between osteoblastic and osteoclastic activity, there are in fact situations where a distortion in the homeostatic balance between osteoblastic and osteoclastic activity is a beneficial consequence. Fracture healing, as well as healing of bone tissue after reconstructive surgery, are two obvious examples where it is desirable to have osteoblastic activity exceed osteoclastic activity. Trauma, whether unintended or intentional, results in increased osteoblastic activity in otherwise healthy and normal bone tissue. Trauma and stress activate the existing osteoblasts and send out signals for the increase in the development of new osteoblasts. Consequently, within a short period of time, there is a vast potential for osteoblastic activity at the trauma site. The cellular activity which activates existing osteoblasts, and increases the development of new osteoblasts, includes the initiation of the synthesis of a new organic matrix when the increased number of osteoblasts are positioned at the trauma site. This organic matrix subsequently serves as the structure into which calcium salts are deposited, creating the newly laid inorganic portion of the bone. In this way, the vital dual organic and inorganic bone tissue components are regenerated at the trauma site. The underlying mechanisms which give rise to the increased osteoblast activity are complex.

Some of the more important aspects of osteoblastic activity will now be described. As already mentioned, osteoblasts facilitate new bone formation or deposition by developing a new organic matrix. This matrix is commonly referred to as the extracellular matrix (ECM). This extracellular matrix serves three critical functions. First, it provides the necessary structure for newly developed osteoblasts to be retained, where their activity is required. Second, the extracellular matrix establishes a network for osteoblast migration, a necessary prerequisite to complete bone regeneration and restructuring at the trauma site. Third, the extracellular matrix constitutes the basic structure in which the mineral or inorganic component of the bone tissue can be deposited. This mineralization only usually occurs in the later stages of tissue repair and replacement and fracture healing. However, it is important to provide the basic structure to allow some mineralization in the early stages of the healing process.

In the early stages of normal bone tissue repair and replacement, as well as, fracture healing, at least two classes of macromolecules play key roles which facilitate the adhesion of bone cells to the organic matrix of bone. This process whereby the cells adhere to the matrix, is a crucial step in the remodeling, repair and replacement of bone tissue. Bone cell adherence is a prerequisite to both bone dissolution and subsequent mineralization in the bone remodeling process as well as complete regeneration of new bone tissue.

One of the key macromolecules having an important role in this adhesion is the integrins, which are receptors on the surface of bone cells. Another key molecule is fibronectin, which is the ligand that interacts with the cell surface integrin. Other ligands can also interact with cell-surface integrins. Examples of ligands that interact with integrins include proteins that may have the RGD (Arg-Gly-Asp) amino acid sequence.

Integrins are receptor proteins consisting of two transmembrane glycoprotein subunits that are non-covalently bound. The subunits are called α and β, and each integrin molecule has one α and one β subunit. Consequently, integrins are referred to as being heterodimeric. There are sixteen known a subunits and eight known β subunits. These α and β subunits are able to combine with each other to form a number of different combinations of complexes. Depending upon the combination of the various α and β subunits, the resulting integrin molecules serve as receptors with specificities for different ligands. The known integrin receptors for collagen are α₁ β₁, α₂β₁ and α₃β₁. The known integrin receptors for fibronectin are α₃ β₁, α₄ β₁, α₅β₁ αᵥβ₁ and α_{III6}β₁.

The major integrins in osteoblasts, α₄β₁ and α₅β₁, recognize the RGD peptide sequence in the appropriate ligand, for example, fibronectin, collagen etc. The overwhelming evidence and laboratory testing indicates that the ligand interaction with the cell surface portion of the integrins is the basis for the adhesion of osteoblasts to the extracellular matrix. In this regard, the relevant portion of the ligand is the adhesive protein RGD-type domain. There may, however, be other isolated and substantially less dominant examples of molecules which form the basis for adhesion, but the ligand interaction with the integrins is believed to be by far the most important. Specific examples of other isolated bases for adhesion possibly include an RGD-resistant cell attachment domain in non-denatured bone sialoprotein. Further, it appears that osteoblastic cells may have an affinity toward collagen and solid substrates that were mediated and facilitated by chondroadherin, yet chondroadherin does not contain an RGD sequence. Despite these examples, the overwhelming evidence supports, as mentioned above, the integrin-type mechanism for adhesion between the osteoblasts and the extracellular matrix.

Fibronectin, which can be generally described as a glycoprotein, operates as a ligand in the integrin/fibronectin relationship. There are two types of fibronectins, namely, plasma and cellular. Plasma fibronectin is derived primarily from hepatocytes, but can also originate from endothelial cells and macrophages. Cellular fibronectin originates from specific cells and likely exists in multiple forms. While plasma and cellular fibronectin are different, their structure and properties are similar.

The structure of the fibronectin molecule can generally be described as asymmetric. It is approximately four times as long as it is wide. The complete molecule is a dimer of similar or identical subunits of approximately 220-kd, with a typical variation of 20-kd either way. The subunits are covalently joined near the carboxyl termini by a disulphide bond. The complete molecule, while asymmetric and elongated, is flexible with globular domains. As mentioned, fibronectin is a glycoprotein, and approximately 5% of the macromolecule is composed of complex oligosaccharides which are linked to asparginal amino acid residues in the chains. The unglycosylated, or protein, portion of the fibronectin is highly susceptible to degradation by the enzyme protease which catalyses the hydrolysis of proteins to amino acids.

The plasma and cellular fibronectin have, as mentioned, certain similar properties and structures, and both affect cellular functions and activities. These functions and activities include: adhesion, morphology, cellular communications, cytoskeletal organization, migration, differentiation, oncogenic transformation, and phagocytosis. While the effects of fibronectin are numerous and important, the most relevant property of fibronectin for the purposes of this invention is the adhesion cellular response to fibronectin. More specifically, the effect of fibronectin upon cellular adhesion is focused on osseous tissue and bone cells.

It is important to note that “adhesion” of bone cells is more involved than simply the interaction of a ligand (fibronectin) with a receptor (integrin). It is believed that at the site of the trauma to the bone, for example a fracture, large numbers of osteoblasts are formed at the surface of the fracture. These newly formed cells begin to migrate as a wave or front which is dependent upon the rate at which they secrete an organic matrix as well as fibronectin which they can use as “handles” as they migrate. The secreted organic matrix (ECM) fills the fracture and in the process the matrix is seeded with the migrating osteoblasts.

In one aspect, the invention provides an initial substitution of the osteoblast “synthesized” matrix with an added, preferably resorbable, matrix to which a ligand, such as fibronectin, is attached. This allows the osteoblasts to move rapidly into the fissure, seed themselves at various sites and initiate many wave fronts from which the de novo synthesis of an organic matrix (ECM) emanating from the newly recruited osteoblasts develop. The invention, therefore, preferably circumvents the rate limiting step of ECM formation by the osteoblasts with a much more rapidly formed or instantaneous matrix (or scaffold) into which the osteoblasts can rapidly migrate to begin many centers of osteoblast-mediated replacement of the scaffold with the newly synthesized ECM. Consequently, adding fibronectin alone, i.e. without the scaffold, would not achieve, at least as effectively, the objective of cellular migration in an orderly fashion into the fracture.

Fibronectin, which is secreted by differentiating or specializing osteoblasts, plays a significant role in bone formation. Fibronectin, in addition to its role as ligand in the osteoblast-organic matrix connection, also appears to regulate the expression of m-RNA associated with the genes that are responsible for osteoblast differentiation. Differentiation is the process by which unspecialized cells become specialized to carry out particular activities and functions. This regulatory aspect of fibronectin in controlling a specific cell function may, at least partially, explain the reason for the rapid flood of new osteoblasts to the site of a trauma to initiate bone remodelling, osseous tissue repair/replacement, and fracture healing.

Another glycoprotein having an important role in osteoblast-organic matrix adhesion is vitronectin. Vitronectin, although an acidic glycoprotein having a pH range between 4.75 and 5.25, has clusters of basic amino acids. The molecular weight of vitronectin ranges from 50-kd to 66-kd, and constitutes a family of similarly structured macromolecules associated with extracellular matrix development, particularly in the arena of atherosclerotic lesions. Circulating vitronectin exists in two forms, namely, a single chain and a double chain. The double chain is made up of two polypeptides, consisting of a larger 65-kd chain and a smaller 10-kd chain, which are held together by a disulphide link. Current research indicates that vitronectin is a class of similarly related macromolecules, yet distinct from each other, but also known and generally characterized as extracellular matrix proteins.

Vitronectin is a multifunctional glycoprotein which plays a role in adhesion, cell regulation, and stabilization of other macromolecules. Insofar as its adhesive properties are concerned, these are similar to the glycoprotein fibronectin, which has been discussed and described above. However, vitronectin has distinctive and unique properties, as will be discussed below, which are quite different, and set it apart from fibronectin. Originating in the liver, vitronectin is deposited at remote sites, especially during extracellular matrix formation during new matrix synthesis. In addition to its origins in the liver, there is also evidence to indicate that vitronectin may also be synthesized locally by bone cells. Most investigations involving vitronectin appear to have been motivated by an effort to understand the clotting process, but vitronectin also plays a key role in the organic matrix of bone remodelling. Like other adhesive proteins, vitronectin contains the RGD peptide sequence (Arg-Gly-Asp), making it a recognizable ligand by integrin receptors. Thus, vitronectin plays an important role as a ligand facilitating adherence between the osteoblasts and the organic matrix in bone regeneration. While vitronectin has many properties, this adhesive property in bone regeneration, as well as its ability to control proteolysis, are significant properties and characteristics for the bone remodelling process.

In addition to vitronectin's role in the adhesion of osteoblasts to the organic matrix, vitronectin, through its ligand interaction with the classic vitronectin receptor, α_{V}β₃, serves as a migratory stimulus for osteoclasts. In order for osteoclasts to carry out their function of resorbing mineralized bone, the osteoclasts are required to be in contact with the bone mineral surface. An attractant, such as vitronectin, in a matrix away from the bone mineral surface, inhibits the bone resorptive quality of osteoclasts.

In addition to attracting osteoclasts away from the bone mineral surface, vitronectin plays an additional role of inhibiting the degradation of the organic matrix. The organic matrix of osseous tissue is susceptible to degradation. Like blood clots, the organic matrix in bone is not a permanent structure. In fact, the same system or process that degrades clots appears to be operative in the bone remodelling processes. The process for clot dissolution involves the proteolysis of fibrin by a serine protease called plasmin. In normal conditions, the concentration of plasmin is extremely low, as it should be, in order to prevent unwanted proteolysis. On the other hand, there are times when a proteolytic event is required, and, in such circumstances, it occurs rapidly and the concentration of plasmin increases to meet this need. This sensitive and quick response control mechanism is a consequence of the ability of plasminogen, the inactive form of plasmin, to be rapidly converted to plasmin by a plasminogen activator. Plasminogen activator is a 72-kd protein having five specific domains. The mechanism whereby plasminogen is converted to plasmin under the influence of the plasminogen activator is well established.

Additional control of the plasminogen activation system exists in the plasminogen activator inhibitors, of which there are two classes. These two classes are SERPINs (serine protease inhibitors) and nexin I. There are two types of inhibitors within the class of SERPINs, namely, PAI-1 and PAI-2. PAI-1 exists in both latent and active forms. The active form is stabilized by interaction with vitronectin, which converts it into the latent form. The PAI-1 binding to vitronectin is believed to be the major, if not exclusive, binding complex for this SERPIN. Consequently, the vitronectin associated with the organic matrix plays a significant role in retaining, restoring and stabilizing PAI-1. Indeed, this is supported by the correlation between the amount of PAI-1 bound to tissues and structures and the amount of vitronectin associated with these elements. In a preferred aspect of the invention, the vitronectin-PAI-1 complex, with its ability to reduce the proteolysis of the organic matrix, supports the net accumulation of the total quantity of bone.

According to an aspect of the invention, there is therefore provided in combination: a structure or framework in which new or regenerated bone tissue can be produced, and the use of certain biological macromolecules in conjunction with the framework. Typically, bone regeneration techniques have centered either on the extracellular matrix, its structure, and the delivery of the matrix, or upon the wide array of biological macromolecules which participate in the bone regeneration process. The invention combines these two aspects, to provide a systematic integration for the efficient and effective remodelling of bone tissue.

Briefly summarized, preferred aspects and embodiments of the invention can be described as follows:

1. Fibronectin facilitates the adhesion of both osteoblasts and osteoclasts.

2. Since osteoblasts lay down new bone and osteoclasts resorb old bone, a system with fibronectin alone would serve only to increase the net turnover rate.

3. Vitronectin attracts osteoclasts away from the bone mineral surface so that the osteoclasts cannot dissolve, or are significantly prevented from dissolving, mineralized bone.

4. Vitronectin makes the PAI more effective.

5. PAI inhibits the degradation of ECM.

6. The combination of fibronectin and vitronectin results in the net accumulation of new bone.

Thus, the invention preferably immobilizes a bioactive (or biologically active) molecule, such as fibronectin, to guide bone-forming cells (osteoblasts) into a three-dimensional framework so that new bone can be formed. By immobilizing vitronectin, bone a resorption is inhibited in as follows: by attracting osteoclasts away from the surface of bone so that the mineral dissolution is prevented or significantly reduced from occurring and inhibiting the proteolysis of the ECM; the vitronectin, after it carries out its function of attracting osteoclasts, is released as the resorbable matrix erodes.

The invention therefore preferably provides a framework or structure in the form of a scaffold for new bone formation, and the immobilization of at least one, and preferably two, bioactive molecules. One bioactive molecule, such as fibronectin, serves as an “attractant” for osteoblast cells, while the other, such as vitronectin, serves to attract osteoclasts away from the bone surface and is also released to inhibit the degradation of the ECM. A regulatory objective can be achieved by purposeful selection of specific bioactive molecules and the equilibrium of the bone turnover is distorted into a desirable direction and outcome.

The invention uses natural constituents, such as collagen, semi-synthetic constituents, such as cross-linked collagen, or synthetic polymers or co-polymers which may be either resorbable or non-resorbable, and which are covalently linked to appropriate biological molecules so that the resulting complex can facilitate cell adhesion, infiltration and migration, and regulate cell, enzymatic or general biologic activity at the implantation site.

The invention combines the features and approaches for replacement, reconfiguring and reconstructing bone tissue. The invention uses new and modified, for example, cross-linking, matrices and scaffolding for repairing tissues in general, and osseous tissue specifically, as well as adhesion and growth promoting proteins. A combination of the scaffolding and the extracellular matrix proteins for osseous tissue repair are used, thereby providing a complete or global approach to bone tissue remodelling through a complete understanding of bone tissue dynamics.

Bone tissue dynamics addresses an apparent dichotomous relationship between the osteoblasts and osteoclasts. Balance between these cells which effect bone forming and bone resorbing, in the absence of a diseased state, results in a homeostatic equilibrium between new bone being formed and existing bone being removed. The invention is based on the premise that not only is a controlled distortion of the equilibrium in favor of new bone formation desirable, whereby osteoblastic activity is enhanced and amplified, but also that the osteoclastic component of the equilibrium process be taken into account to ensure optimal bone tissue regeneration. Facilitating osseous tissue healing resulting from fractures, reconstructions, or replacements of bone losses is the net consequence of increased bone formation with the simultaneous decrease of bone resorption. This is achieved by stimulating osteoblastic activity, inhibiting osteoclastic activity, or both. The invention specifically employs as a tool in the facilitation of osseous tissue repair the regulation of osteoclastic activity to control and/or reduce bone resorption during bone regeneration which has become necessary through trauma or disease.

The regulation of and/or reduction in the impact of osteoclastic activity is necessary for effective bone regeneration in view of the sequence of events related to osteoblastic activity immediately after a traumatic event such as fracture or a mechanical manipulation of osseous tissue which occurs in reconstructive surgery. As a direct consequence of a traumatic event to osseous tissue, osteoblastic activity is naturally elevated. Consequently, in response to the trauma, the equilibrium is naturally distorted in favor of new bone formation. The osteoblastic activity and effect is enhanced and hastened by the concomitant inhibiting or impeding of the bone resorption process by the regulation of the osteoclastic cells.

According to a further aspect of the invention, there is provided a system and method for regulating both osteoblastic and osteoclastic activities simultaneously. Preferably, the system and method employs the glycoproteins fibronectin and/or vitronectin, both of which have cell adhesion properties, and these are covalently anchored to a biodegradable resorbable component. Preferably, these adhesion glycoproteins are secure and will remain in place until the scaffold disintegrates. Conveniently, they are not mere additives which will be removed in the short term. The effectiveness of the added adhesive glycoproteins is a function of the dissolution or disappearance rate of the scaffold. Preferably, therefore, the degree of polymerization of the synthetic scaffold is appropriately selected so that differential disappearance rates can be achieved for the covalently anchored fibronectin and vitronectin. Thus, for example, by carefully selecting the synthetic scaffold to have properties enabling it to last longer, the fibronectin and vitronectin adhesive proteins will remain at the site of bone regeneration longer. This in turn facilitates the creation and preservation of the extracellular matrix upon which the inorganic portion of the bone can grow by the appropriate deposit of minerals forming the hydroxyapatite, and other essential components of the inorganic bone tissue.

While vitronectin has traditionally been considered a cell adhesive protein, it has a particularly useful application within the context of the present invention in view of its cellular as well as its extracellular regulating properties. Accordingly, a further aspect of the invention includes the use of vitronectin in the regeneration of bone tissue, the vitronectin having cell adhesive properties facilitating the development of the extracellular matrix, as well as its cellular and extracellular regulating properties.

With respect to the non-adhesive, or cell regulating, properties of the vitronectin glycoprotein, the most important such property is the ability of vitronectin to inhibit the proteolysis of the extracellular matrix. In this invention, the vitronectin preferably interacts with the plasminogen activator inhibitor (PAI) to stabilize it (i.e. convert it from its active to its latent form) and make it available to inhibit the plasminogen-based degradation of the extracellular matrix. The plasminogen activator inhibitor appears to be short-lived. To ensure that there is sufficient vitronectin available to sustain its function of maintaining supplies of plasminogen activator inhibitor, the covalently bound vitronectin is chemically connected to a collection of varying molecular weight synthetic polymers, so that upon dissolution, at varying rates, of the synthetic polymer scaffold, there is a constant supply of vitronectin available to stabilize the plasminogen activator inhibitor. Thus, the plasminogen activator inhibitor can be stored and made available for release to prevent the plasminogen activator from converting the plasminogen to plasmin. This is a desirable consequence in view of the fact that plasmin results in the proteolysis of fibrin, and therefore contributes towards the degradation of the developing extracellular matrix which is being created, negatively impacting the effective regeneration of bone. The use of vitronectin, not only for its adhesive properties, but also for its ability to stabilize and make available the plasminogen activator inhibitor, makes the appropriate use of vitronectin in bone regeneration a powerful tool.

Like fibronectin, vitronectin operates as a ligand, extending between the integrin receptor forming part of the osteoblast cell and the organic matrix. In its function as stabilizer of the plasminogen activator inhibitor, the vitronectin may also be covalently bound in a chemical connection to synthetic polymer scaffolds. Further, the vitronectin may be connected to a number of such synthetic polymer scaffolds having varying molecular weights, the different synthetic polymer scaffolds being designed to dissolve at different rates. Thus, the fairly constant dissolution over a period of time of the synthetic polymer scaffolds has the effect of releasing vitronectin in a steady supply making it available to stabilize the plasminogen activator inhibitor. This stabilization and availability of the plasminogen activator inhibitor ultimately protects the extracellular matrix by controlling the hydrolytic or catalytic activity of the proteolytic enzyme plasmin. The preservation of the extracellular matrix provides the necessary opportunity for the building thereon of the inorganic mineralized components of the bone tissue, thus playing an important role in expediting bone regeneration.

The covalent binding of biologic macromolecules to a resorbable matrix is an important aspect of the invention. Such covalent binding allows cellular infiltration by the host's own cells in vivo, having the advantage of assisting in the replacement, reconfiguring or reconstruction of the tissue as the scaffold disappears. Thus, the scaffold provides not only an initial substrate or basis used by the host's own cells to initiate the bone regeneration process, but also locks up macromolecules which play a role in bone regeneration, and which are released as the scaffold degenerates. The description given above with respect to the vitronectin glycoprotein is a good example of the advantageous effects of covalently binding these biologic macromolecules to the resorbable matrix.

The covalent binding of biologic macromolecules to a resorbable matrix has other beneficial effects. First, the type of matrix selected, or the types of matrices combined to form a scaffold, can be chosen according to predetermined criteria so as to control and regulate the resorbing or dissolution rates. Additionally, the matrix can be constructed so as to include cell regulators known to have specific effects on different cell types. Ultimately, therefore, the balance between osteoblastic and osteoclastic activity during the repairing process can be regulated and controlled so as to have an optimal effect on the bone regeneration process.

### BRIEF DESCRIPTION OF THE DRAWINGS

FIG. 1 is a tree chart showing, in diagrammatic form, some of the various components constituting bone tissue;

FIG. 2 is a diagrammatic representation of an organic matrix; and

FIG. 3 is a diagram showing the relationship of plasmin with its activators and inhibitors.

### DETAILED DESCRIPTION OF THE PREFERRED EMBODIMENTS

In one aspect, the invention comprises a matrix polymer for use as a scaffold and one or more biologically active molecules connected to the matrix polymer scaffold.

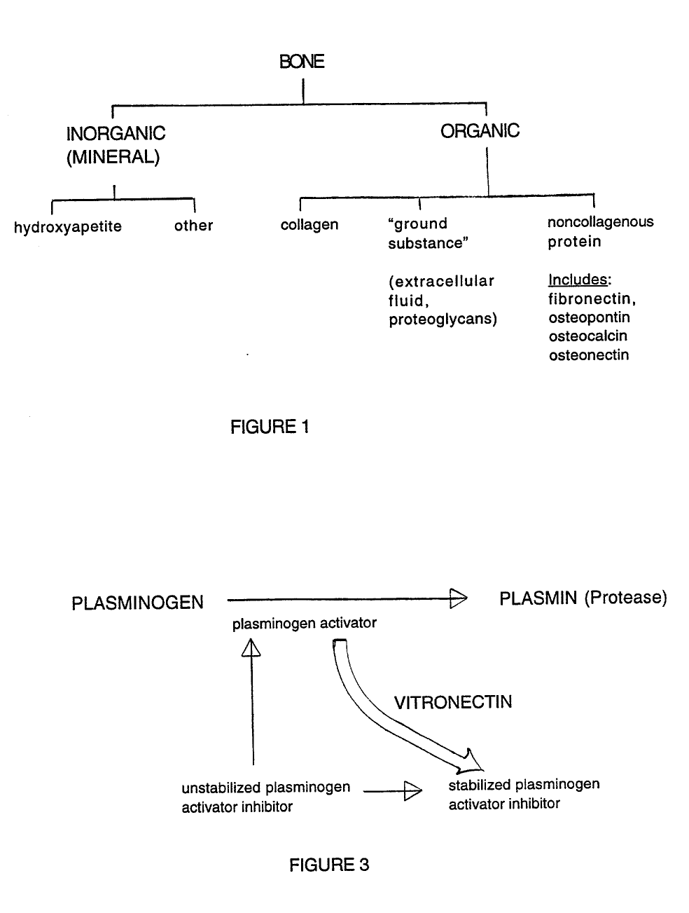

FIG. 1 is a chart showing the general composition of bone tissue. As described above, bone has two essential components, namely, an inorganic mineral component, which forms the dominant portion of bone, and a smaller, but very highly functional, organic component. The mineral portion of bone consists principally of hydroxyapatite, a complex crystalline form of calcium and phosphate ions. There are other components, which are less crystalline in form, which tend to give bone its amorphous appearance. These mineral components include magnesium, sodium, potassium and other less prevalent cations. The organic portion of bone can be divided into three main sections, namely, the collagen fiber portion consisting of 90-95% of the organic portion, with the remaining 5-10% being comprised of “ground substance” and non-collagenous proteins. The ground substance has as its major components the extracellular fluid and the proteoglycans, a high molecular weight polyanionic substance covalently linked by numerous heteropolysacharride side chains to a polypeptide chain backbone. The non-collagenous proteins in the organic component play a very important role in bone structure, as well as in the regenerative process for adhesive and regulatory functions. The non-collagenous proteins includes fibronectin, osteopontin, osteocalcin, osteonectin, thrombospondin and other less characterized proteins. These proteins also play an important role in intra- and inter-cellular communications.

The scaffold may be comprised of natural polymers or synthetic polymers. The natural polymers may be collagens, hyaluronic acid, heparin, proteoglycans, glycoproteins and lipopolysacharrides. Other natural scaffold polymers which may be used in accordance with the invention include demineralized bone, cross-linked and derivatized natural polymers and materials that contain proteoglycans.

With respect to the synthetic scaffold polymers, these may be either resorbable polymers, or somewhat less resorbable polymers. The resorbable polymers include the polyester and polyamide polymers, homo- and heteropolymers, using, but not limited to, the following monomeric units and combinations thereof: glycolic, lactic, ε-caprolactone or any organic carboxylic acid including mono-carboxylic acid, dicarboxylic acid etc. containing one or more additional functional groups that could form esters or amides. In a heteropolymer, at least one of the monomers should preferably contain a functional group that can form an ester or an amide while the other monomer need not have this functional group.

Less resorbable synthetic scaffold polymers which may be used in constructing the scaffold include: polyanhydrides, polyurethanes, polyacrylonitriles, polyvinyl alcohol, polymethylmethacrylate and polyphosphazenes.

With respect to the biologically active molecules connected to the matrix polymer scaffold, these can be selected from a wide variety of active molecules. Preferred biologically active molecules include the following:

proteoglycans;

fibronectin and fibronectin fragments;

vitronectin and vitronectin fragments;

collagen and collagen fragments;

heparin and heparin fragments;

von Willebrand factor

bone sialoprotein

osteopontin

osteonectin

osteocalcin

selectin and selectin fragments;

proteins and peptides that facilitate cell adhesion (including cyclic versions):

RGD-Type (Arg-Gly-Asp) and RGDS-Type (Arg-Gly-Asp-Ser) e.g., RGDC (Arg-Gly-Asp-Cys), RGDV (Arg-Gly-Asp-Val), RGES (Arg-Gly-Glu-Ser), GRGDS (Gly-Arg-Gly-Asp-Ser), GRADSP (Gly-Arg-Ala-Asp-Ser-Pro), KGDS (Lys-Gly-Asp-Ser), GRGDSP (Gly-Arg-Gly-Asp-Ser-Pro), GRGDTP (Gly-Arg-Gly-Asp-Thr-Pro), GRGES (Gly-Arg-Gly-Glu-Ser), GRGDSPC (Gly-Arg-Gly-Asp-Ser-Pro-Cys), GRGESP (Gly-Arg-Gly-Glu-Ser-Pro), SDGR (Ser-Asp-Gly-Arg), YRGDS (Tyr-Arg-Gly-Asp-Ser), GQQHHLGGAKQAGDV (Gly-Gln-Gln-His-His-Leu-Gly-Gly-Ala-Lys-Gln-Ala-Gly-Asp-Val), GPR (Gly-Pro-Arg) etc.;

GHK-Type (Gly-His-Lys)etc.;

YIGSR-Type (Tyr-Ile-Gly-Ser-Arg); PDSGR (Pro-Asp-Ser-Gly-Arg); CDPGYIGSR (Cys-Asp-Pro-Gly-Tyr-Ile-Gly-Ser-Arg); laminin or laminin-fragment etc.;

LCFR-Type (Leu-Cys-Phe-Arg) etc.;

EIL-Type e.g., EILDV (Glu-Ile-Leu-Asp-Val), EILDVPST (Glu-Ile-Leu-Asp-Val-Pro-Ser-Thr), EILEVPST (Glu-Ile-Leu-Glu-Val-Pro-Ser-Thr) etc.;

LDV-Type LDVPS (Leu-Asp-Val-Pro-Ser), LDV-NH2 (Leu-Asp-Val-NH2) etc.;

synthetic peptides containing the RGD, RGDS, GHK, LCFR or YIGSR sequence of amino acids;

osteonectin and SPARC (Secreted Protein Acidic and Rich in Cysteine);

osteopontin;

collagens, Type I and Type II;

von Willebrand Factor (a glycoprotein that facilitates adhesion of cells to structures. It has an ability to link to cells and thus the potential of being a ligand for cell surface receptors of osteoblasts);

bone sialoprotein;

thrombospondin;

osteocalcin;

cytomodulin;

bone morphogenetic proteins (BMPs);

tenascins;

fibrinolysis inhibiting factor;

growth factors e.g., Platelet Derived Growth; Factors (PDGF), Insulin-Like Growth Factors (IGFs), etc.

antibodies to cell surface components e.g., β-1; integrin antibody;

plasminogen activator inhibitors (PAIs);

protease inhibitors; and

metalloprotease inhibitors.

(In the above description, the conventional notation is followed whereby: R=Arg, G=Gly, D=Asp, S=Ser, C=Cys, V=Val, E=Glu, A=Ala, P=Pro, K=Lys, T=Thr, Y=Tyr, Q=Gln, H=His, L=Leu, I=Ile and F=Phe.)

It is advantageous also to use “spacer” or linking molecules and such linking molecules may comprise homobifunctional or heterobifunctional cross-linking agents, or polymeric spacers, such as polyethoxylates, polyethylene glycol, polysorbitals, or other appropriate molecules. Preferably, the spacer sets the bioactive molecule, for example fibronectin, out and away from the matrix while still holding and retaining the bioactive molecule as part of the matrix. This will allow the cells to interact with the bioactive molecule without any, or significantly reduced, hindrance from the matrix structures. Spacers also facilitate freedom of movement of the bioactive molecule in a three dimensional way (latitude, longitude and altitude). Movement is in a plane parallel to the surface plane created by the matrix which is a distance from the surface plane of the matrix, equal to the length of the spacer. Another advantage of having a spacer is that, depending on the chemical structure of the spacer, i.e. whether or not it has double bonds, the spacer may provide rotational freedom for the bioactive molecule.

The synthetic, resorbable polymer scaffold preferably has certain characteristics and properties to make it more effective in the context of bone regeneration. Either pure glycolide, pure lactide or ε-caprolactone, or co- and tri-polymeric mole fractions of the monomers are preferably used, and can be purchased from various suppliers, including, for example, Birmingham Polymers, Inc., of Birmingham, Ala. Preferably, the preparation of these materials or other hydroxy esters is such that a product, having either free alcohol or carboxyl functional groups results. It is important, in one aspect of the invention, to control the degree or extent of polymerization relative to two important variables. These are, on the one hand, solubility or degradability, and, on the other hand, the ratio of the free functional groups.

The degradation time line of the synthetic scaffold is more rapid where lower molecular weight polymers are used. The ratio of monomers in the final co- or tri-polymer product also affects the resorbability. In addition, the lower the molecular weight, the more free functional groups are available per total mass of polymer. This is significant since it affects the number of available sites to which biologically active molecules such as fibronectin and vitronectin are connected. As such, controlling the molecular weight of the synthetic polymer effectively controls the in vivo resorbability as well as the amount of biologically active moles that can be attached to the scaffold.

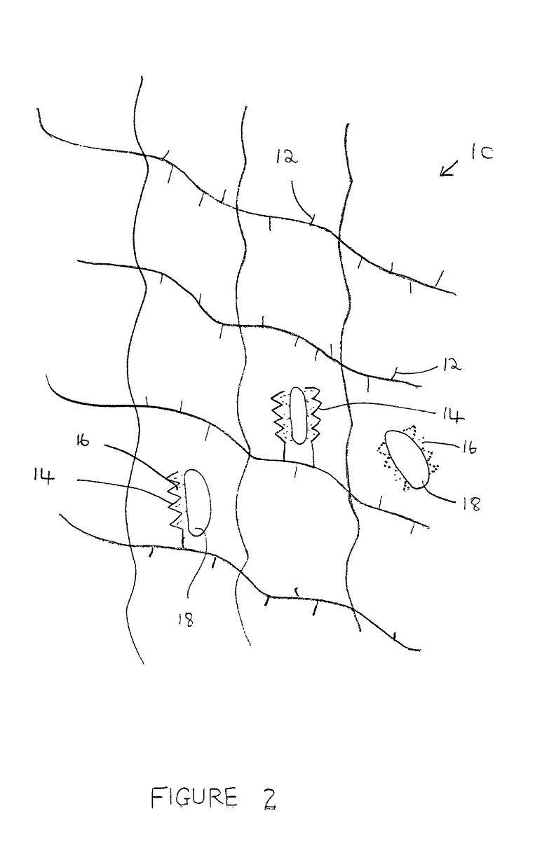

Reference is now made to FIG. 2 of the drawings, which is a diagrammatic representation of an organic matrix. In FIG. 2, the organic matrix 10 has thereon a plurality of binding sites 12 for biologically active molecules. A biologically active molecule 14, an example of which is a ligand, fibronectin, is attached to the binding site 12. The biologically active molecule binds to a cell surface receptor 16 of an osteoblast 18, thereby providing “adhesion” or attachment for the osteoblast molecules within the organic matrix 10.

With reference to FIG. 3, there is shown a diagrammatic representation of plasmin and its activator, inhibitor, and inactive form. Plasmin is an active enzyme that attacks and dissolves fibrin material. Plasminogen is the inactive form of plasmin, and is activated by the plasminogen activator. Thus in the presence of the plasminogen activator, plasminogen transforms from its inactive form to its active form of plasmin. During bone regeneration, it is desirable to reduce the amounts of plasmin at the regeneration site in order to provide as much opportunity as possible for the organic matrix to be created and grow, thus forming the basis for the organic material of bone. For this purpose, it is desirable to have quantities of stabilized and/or unstabilized plasminogen activator inhibitor to prevent plasmin formation from its inactive form plasminogen. As seen in the diagram of FIG. 3, plasminogen activator inhibitor prevents the formation of plasmin from plasminogen. As described above and schematically illustrated in FIG. 3, the ability of vitronectin to bind to the plasminogen activator inhibitor, and release the inhibitor as the vitronectin is released from the degrading organic matrix, assists in the regenerative bone process.

Various examples showing application of the invention are set forth below. These examples are meant to be illustrative only, and are not in any way intended to limit the scope of the invention.

The examples below are divided into two groups, the first relating to synthesis of the biological molecules and scaffolding, while the second group of examples relates to the in vivo application of the invention.

### EXAMPLES

### Example 1

Modification of the Synthetic Resorbable Polymer

Carboxyl-terminal polyester e.g., poly(L-lactic acid), polyglycolic acid, polylactin, poly(DL-lactic-co-glycolic acid), poly(ε-caprolactone), poly(L-lactic acid-co-caprolactone), poly(glycolic acid-co-caprolactone) etc. of varying mole-percent compositions of monomers and molecular weights are derivatized at the free carboxyl groups using a modification of the procedure of Williams et al. (1981). In this procedure 1-ethyl-3-[-3-dimethylaminopropyl]-carbodiimide (EDC) serves as the coupling agent. The EDC-activated carboxyl group of the synthetic resorbable polymer is coupled to the free amine groups associated with a biologically active polypeptide and polypeptide fragments. (Williams, A. and Ibrahim, E. A. “A Mechanism Involving Cyclic Tautomers for the Reaction with Nucleophiles of the Water-Soluble Peptide Coupling Agent 1-Ethyl-3-[-3-Dimethylaminopropyl]-Carbodiimide (EDC).” J. Am. Chem. Soc. 103, 7090-7095(1981).)

As an alternative to the modification procedure set out above, the carboxyl groups on the synthetic resorbable polymer are converted by a reduction reaction to aldehydes. The aldehydes on the synthetic resorbable polymer are then reacted with the free amine groups on the biologically active peptides through a Scoff base reaction.

### Example 2

Modification of the Biologically Active Peptide

Using procedures similar to Example 1 set out above, as well as the general approach thereof, the biologically active peptide is modified and connected to the free carboxyl group of the synthetic resorbable polymer.

### Example 3

Modification of Both the Synthetic Resorbable Polymer and the Biologically Active Peptide

Under certain circumstances, it may be advantageous to modify both the synthetic resorbable polymer and the biologically active polypeptide prior to the derivatization step described in Example 1 above. Whether or not the synthetic resorbable polymer and the biologically active polypeptide are modified prior to the derivatization step will usually depend upon the basic properties or structure of the biologically active polypeptide. In any event, when both polymer and active peptide are modified, the approach as set out in Examples 1 and 2 describing such modification would typically be used.

### Example 4

Derivatization of a Synthetic Resorbable Polymer with a Biologically Active Peptide without Prior Modification of Either

The free hydroxyl groups of serine or hydroxy proline amino acid residues in a biologically active polypeptide can be directly esterified to the free carboxyl groups of the synthetic resorbable polymer to form the derivatized polymer-peptide complex.

### Example 5

Use of Synthetic Resorbable Polymer with Free Hydroxyl Groups

The process and procedures used in Examples 1 to 4 are employed to produce a polymer-peptide complex. However, in this case, the starting polymeric material is one that has free hydroxyl groups, rather than free carboxyl groups.

### Example 6

Use of a Connecting Link or “Spacer” Molecule Between the Synthetic Resorbable Polymer and the Biologically Active Peptide

In this example, a bridge, or connecting link or “spacer”, is attached either to the synthetic resorbable polymer or the biologically active peptide prior to the derivatization step. In this process, the biologically active peptide is connected to the synthetic resorbable polymer in an indirect fashion, using a molecule that is, at least, bi-functional. The bi-functional molecule has one functional group which is connected to the synthetic resorbable polymer, with the other functional group of this “spacer” molecule being connected to the biologically active peptide.

The examples set out above relate to the synthesis of the resorbable polymer, the biologically active peptide, or both. The examples set out below describe the in vivo application based on the synthesis described above.

### Example 7

Application of the Synthetic Resorbable Polymer—Biologically Active Peptide Complex Composition

After a suitable clean-up procedure following the derivatization reaction, the polymer-peptide complex is implanted or molded onto an osseous tissue surface or injected into a fracture or an eroded portion of osseous tissue.

### Example 8

Composition to Stimulate New Bone Synthesis

A synthetic resorbable matrix is derivatized with biologically active molecules that increase both number and activity of osteoblastic cells. These biologically active molecules include one or more of the following molecules connected, as described in Examples 1 to 6, to the resorbable polymer: fibronectin, fibronectin fragments, collagen, collagen binding fragments, heparin, heparin binding fragments, selectin, selectin binding fragments, proteins and peptides that facilitate cell adhesion and antibodies to cell surface components.

This composition is typically used in instances where bone remodelling is desired, for example, such an in reconstructive surgery, implantation of prosthetic devices, replacement of excised bone fragments, and other such reconstructive procedures.

### Example 9

Composition to Inhibit Bone Resorption and Bone Loss

A synthetic resorbable matrix is derivatized with biologically active molecules that inhibit the erosion or dissolution of the extracellular matrix of bone, either directly or by rendering osteoclasts less effective. These biological molecules, connected to the matrix as described in Examples 1 to 6 above, include one or more of the following: vitronectin, plasminogen activator-inhibitor, metalloprotease inhibitor or other protease inhibitors.

This composition set out in the present example is typically used to counteract excessive bone resorption in conditions or situations such a periodontal disease, repair of portions of extensive fractures, and other such situations.

### Example 10

Composition to Reduce the Healing Time of Bone Fractures and Minor Bone Fissures

A first sample of synthetic resorbable matrix is derivatized with biologically active molecules that increase both numbers and activity of osteoblasts at the fracture site. The biologically active molecules will include those described in Example 8 above. A second sample of synthetic resorbable matrix is derivatized with biologically active molecules that inhibit the erosion or dissolution of the extracellular matrix of bone, whether directly, or by rendering osteoclasts less effective. Such a composition is described in Example 9 above. Thus, by combining a composition to stimulate new bone synthesis, with a further composition to inhibit bone resorption and bone loss, the healing time of bone fractures and minor bone fissures can be reduced.

The two samples of the derivatized synthetic resorbable matrix in this example may be combined in various ratios and proportions to achieve the desired healing rate. Further, adjustment of the healing rate may be controlled by the selection of the various biologically active molecules that act simultaneously at the same location. In addition, by selecting the appropriate molecular weight of the synthetic resorbable polymer, additional control of the healing rate may be achieved.

1. A composition for modulating bone regeneration, the composition comprising:
a matrix selected from the group consisting of glycolic acid, lactic acid, collagen, demineralized bone, or a combination thereof;
a first biologically active molecule comprising a fibronectin to facilitate osteoblast activity for promoting an increase in bone formation, the first biologically active molecule being attached to at least a portion of the matrix; and
a second biologically active molecule comprising a vitronectin selected for its ability to attract osteoclasts and produce an inhibiting effect on osteoclast activity to thereby promote a decrease in bone resorption, the second biologically active molecule being attached to at least a portion of the matrix substrate.

2. A composition as claimed in claim 1 wherein the fibronectin comprises an amino acid binding sequence that binds to the osteoblasts.

3. A composition as claimed in claim 2 wherein the amino acid binding sequence is selected from one or more of the group consisting of:
RGD-Type (Arg-Gly-Asp) and RGDS-Type (Arg-Gly-Asp-Ser), RGDC (Arg-Gly-Asp-Cys), RGDV (Arg-Gly-Asp-Val), RGES (Arg-Gly-Glu-Ser), GRGDS (Gly-Arg-Gly-Asp-Ser), GRADSP (Gly-Arg-Ala-Asp-Ser-Pro), KGDS (Lys-Gly-Asp-Ser), GRGDSP (Gly-Arg-Gly-Asp-Ser-Pro), GRGDTP (Gly-Arg-Gly-Asp-Thr-Pro), GRGES (Gly-Arg-Gly-Glu-Ser), GRGDSPC (Gly-Arg-Gly-Asp-Ser-Pro-Cys), GRGESP (Gly-Arg-Gly-Glu-Ser-Pro), SDGR (Ser-Asp-Gly-Arg), YRGDS (Tyr-Arg-Gly-Asp-Ser), GQQHHLGGAKQAGDV (Gly-Gln-Gln-His-His-Leu-Gly-Gly-Ala-Lys-Gln-Ala-Gly-Asp-Val), GPR (Gly-Pro-Arg);
GHK-Type (Gly-His-Lys);
YIGSR-Type (Tyr-Ile-Gly-Ser-Arg); PDSGR (Pro-Asp-Ser-Gly-Arg); CDPGYIGSR (Cys-Asp-Pro-Gly-Tyr-Ile-Gly-Ser-Arg); laminin or laminin-fragment;
LCFR-Type (Leu-Cys-Phe-Arg);
EIL-Type, EILDV (Glu-Ile-Leu-Asp-Val), EILDVPST (Glu-Ile-Leu-Asp-Val-Pro-Ser-Thr), EILEVPST (Glu-Ile-Leu-Glu-Val-Pro-Ser-Thr);
LDV-Type LDVPS (Leu-Asp-Val-Pro-Ser), LDV-NH₂ (Leu-Asp-Val-NH₂);
synthetic peptides containing the RGD, RGDS, GHK, LCFR or YIGSR sequence of amino acids;
osteonectin and SPARC (Secreted Protein Acidic and Rich in Cysteine);
osteopontin;
collagens, Type I and Type II;
von Willebrand Factor;
bone sialoprotein;
thrombospondin;
osteocalcin;
cytomodulin;
bone morphogenetic proteins (BMPs);
tenascins;
fibrinolysis inhibiting factor;
growth factors, Platelet Derived Growth Factors (PDGF), Insulin-Like Growth Factors (IGFs); and
antibodies to cell surface components, β-1; integrin antibody.

4. A composition as claimed in claim 1 wherein the vitronectin comprises an amino acid binding sequence that binds to the osteoclasts.

5. A composition as claimed in claim 4 wherein the amino acid binding sequence is selected from one or more of the group consisting of:
RGD-Type (Arg-Gly-Asp) and RGDS-Type (Arg-Gly-Asp-Ser), RGDC (Arg-Gly-Asp-Cys), RGDV (Arg-Gly-Asp-Val), RGES (Arg-Gly-Glu-Ser), GRGDS (Gly-Arg-Gly-Asp-Ser), GRADSP (Gly-Arg-Ala-Asp-Ser-Pro), KGDS (Lys-Gly-Asp-Ser), GRGDSP (Gly-Arg-Gly-Asp-Ser-Pro), GRGDTP (Gly-Arg-Gly-Asp-Thr-Pro), GRGES (Gly-Arg-Gly-Glu-Ser), GRGDSPC (Gly-Arg-Gly-Asp-Ser-Pro-Cys), GRGESP (Gly-Arg-Gly-Glu-Ser-Pro), SDGR (Ser-Asp-Gly-Arg), YRGDS (Tyr-Arg-Gly-Asp-Ser), GQQHHLGGAKQAGDV (Gly-Gln-Gln-His-His-Leu-Gly-Gly-Ala-Lys-Gln-Ala-Gly-Asp-Val), GPR (Gly-Pro-Arg);
GHK-Type (Gly-His-Lys);
YIGSR-Type (Tyr-Ile-Gly-Ser-Arg); PDSGR (Pro-Asp-Ser-Gly-Arg); CDPGYIGSR (Cys-Asp-Pro-Gly-Tyr-Ile-Gly-Ser-Arg); laminin or laminin-fragment;
LCFR-Type (Leu-Cys-Phe-Arg);
EIL-Type, EILDV (Glu-Ile-Leu-Asp-Val), EILDVPST (Glu-Ile-Leu-Asp-Val-Pro-Ser-Thr), EILEVPST (Glu-Ile-Leu-Glu-Val-Pro-Ser-Thr);
LDV-Type LDVPS (Leu-Asp-Val-Pro-Ser), LDV-NH₂ (Leu-Asp-Val-NH₂);
synthetic peptides containing the RGD, RGDS, GHK, LCFR or YIGSR sequence of amino acids;
osteonectin and SPARC (Secreted Protein Acidic and Rich in Cysteine);
osteopontin;
collagens, Type I and Type II;
von Willebrand Factor;
bone sialoprotein;
thrombospondin;
osteocalcin;
cytomodulin;
bone morphogenetic proteins (BMPs);
tenascins;
fibrinolysis inhibiting factor;
growth factors, Platelet Derived Growth Factors (PDGF), Insulin-Like Growth Factors (IGFs); and
antibodies to cell surface components, β-1; integrin antibody.

6. A composition as claimed in claim 1 further comprising spacer molecules between the matrix and the first and/or second biologically active molecules.

7. A composition as claimed in claim 6 wherein the spacer molecules are selected from homo-bifunctional or hetero-bifunctional cross-linking agents.

8. A composition as claimed in claim 6 wherein the spacer molecules comprise polymeric spacers.

9. A composition as claimed in claim 8 wherein the polymeric spacers are selected from the group consisting of: polyethoxylates, polyethylene glycol, polysorbitols, and combinations thereof.

10. A method of making a composition for modulating bone regeneration comprising:
selecting a matrix from the group consisting of glycolic acid, lactic acid, collagen, demineralized bone, or a combination thereof;
attaching a first biologically active molecule comprising a fibronectin to facilitate osteoblast activity for promoting an increase in bone formation to at least a portion of the matrix; and
attaching a second biologically active molecule comprising a vitronectin selected for its ability to attract osteoclasts and produce an inhibiting effect on osteoclast activity to thereby promote a decrease in bone resorption to at least a portion of the matrix substrate.

11. A composition for treating bone tissue comprising:
a matrix capable of forming a scaffold;
a first biologically active molecule comprising a fibronectin to facilitate osteoblast activity attached to at least a portion of the matrix; and
a second biologically active molecule comprising a vitronectin selected for its ability to attract osteoclasts attached to at least a portion of the matrix substrate..

12. A composition as claimed in claim 11 wherein the matrix forming a scaffold comprises a biodegradable or resorbable component, and the vitronectin is covalently anchored to the biogradable component.

13. A composition as claimed in claim 11 wherein the matrix forming a scaffold comprises a biodegradable or resorbable component, and the fibronectin is covalently anchored to the biogradable component.

14. A composition as claimed in claim 11 wherein the matrix is selected so that it has a differential disappearance rate which is a function of the polymerization thereof.

15. A composition as a claimed in claim 11 wherein the matrix is selected to control the time for degradation of the organic matrix.

16. A composition for inhibiting proteolysis of extracellular matrix in bone tissue, the composition comprising:
a biodegradable matrix forming a scaffold;
vitronectin attached to the matrix for release therefrom as the matrix degrades.

17. A composition as claimed in claim 16 wherein the matrix further comprises one or more from the group consisting of plasminogen activator inhibitor, metalloprotease inhibitor, protease inhibitors and combinations thereof, attached to the matrix.

18. A method of treating bone tissue comprising:
forming an inorganic matrix having a predetermined dissolution rate;
attaching vitronectin proteins to the matrix whereby vitronectin is released from the matrix in a controlled manner according to the rate of dissolution of the inorganic matrix.

19. A method as claimed in claim 18 wherein the vitronectin released from the matrix is bound to a plasminogen activator inhibitor, the plasminogen activator inhibitor being released from the vitronectin as the inorganic matrix dissolves to thereby reduce the production of proteolytic plasmin.
